(19) United States
(12) Patent Application Publication
(10) Pub. No.: US 2025/0064302 A1
(43) Pub. Date: Feb. 27, 2025

(54) UMBILICUS CABLES FOR USE WITH MEDICAL DEVICES

(71) Applicant: Boston Scientific Scimed, Inc., Maple Grove, MN (US)

(72) Inventors: Kirsten VIERING, Newton, MA (US); Louis J. BARBATO, Franklin, MA (US)

(73) Assignee: Boston Scientific Scimed, Inc., Maple Grove, MN (US)

(21) Appl. No.: 18/812,361

(22) Filed: Aug. 22, 2024

Related U.S. Application Data
(60) Provisional application No. 63/578,165, filed on Aug. 23, 2023.

(51) Int. Cl. A61B 1/00; A61B 1/06

(52) U.S. Cl. CPC A61B 1/00114; A61B 1/0004; A61B 1/00105; A61B 1/00124; A61B 1/00128; A61B 1/0669; A61B 1/00066; A61B 1/00119

(57) ABSTRACT

A medical device may comprise a first part. The first part of the medical device may have a handle, a shaft attached to the handle, and a distal assembly at a distal end of the shaft. The medical device may also comprise an umbilicus releasably attached to the handle of the first part.

### CROSS-REFERENCE TO RELATED APPLICATIONS

This application claims the benefit of priority under 35 U.S.C. § 119 from U.S. Provisional Application No. 63/578,165, filed Aug. 23, 2023, which is incorporated by reference herein in its entirety.

### TECHNICAL FIELD

Various aspects of this disclosure relate generally to medical device components, assemblies, and associated methods. In particular, aspects of this disclosure pertain to reusable umbilicus cables for use with medical devices, among other aspects.

### BACKGROUND

A medical device (e.g., a duodenoscope, an endoscope, a bronchoscope, an endoscopic ultrasonography (“EUS”) scope, etc.) may be used for an endoscopy procedure, where the medical device comes in contact with a patient. After the endoscopy procedure, the medical device may be cleaned in order to be used for another endoscopy procedure. Alternatively, the medical device may be a single-use device to minimize cross-contamination between patients, where after an endoscopy procedure, the medical device may be thrown out and a new medical device may be used for the next endoscopy procedure.

### SUMMARY

Each of the aspects disclosed herein may include one or more of the features described in connection with any of the other disclosed aspects.

In an example, a medical device may comprise a first part having a handle, a shaft attached to the handle, and a distal assembly at a distal end of the shaft; and an umbilicus releasably attached to the handle of the first part.

Any of the devices disclosed herein may include any of the following features, additionally or alternatively, in any combination. The umbilicus may include a tether that branches off from a main body of the umbilicus. The tether may include at least one button at an end of the tether furthest from the main body of the umbilicus, wherein the at least one button is configured to control an imaging function of the medical device. An end of the tether furthest from the main body may include an engagement feature for securably and releasably engaging the handle of the first part. The engagement feature may be configured to securably and releasably engage multiple different positions of the handle. The tether may have a shape or a protrusion to engage a correspondingly-shaped portion in a body of the handle, wherein the protrusion may be a snap-fit engagement, a friction-fit engagement, or a relative sliding engagement that may securely hold the tether to the handle and permit release after use. The engagement feature may include a magnet that attaches to the handle via a metal feature of the handle. The engagement feature may include a c-shaped connector. The umbilicus may be configured to connect to the handle at a connection point via a mating, physical and electrical connection that securely and releasably attaches the umbilicus to the handle. The umbilicus may include a handle connection end, wherein the handle connection end includes an umbilicus connector, a flexible printed circuit board, and a heat sink. The umbilicus may include a handle connection end converter located within the handle connection end, wherein the handle connection end converter may convert a plurality of electrical signals into a plurality of light signals for transmission through circuitry in a main body of the umbilicus to a controller connection end. The umbilicus may include a controller connection end converter located within a controller connection end, wherein the controller connection end converter may convert a plurality of light signals into a plurality of electrical signals for transmission to a controller. The medical device may further comprise one or more fluidics tubing removably attached to the umbilicus via a clip, wherein the one or more fluidics tubing is fixed to the handle. The umbilicus may include a flexible circuit located in an end of the umbilicus that is closest to the handle when connected. The flexible circuit may include at least one umbilicus connector connection attached to at least one electrical connection of the handle, at least one umbilicus cable connection attached to a cable within the umbilicus, and an outer layer that includes at least one additional flexible circuit.

In another example, an umbilicus may comprise a main body that includes circuitry for transmitting power, imaging signals, and light or light signals; a handle connection end at a first end of the main body, wherein the handle connection end is configured to releasably connect to a medical device handle; and a controller connection end at a second end of the main body opposite the handle connection end, wherein the controller connection end is configured to releasably connect to a controller.

Any of the devices described herein may include any of the following features, additionally or alternatively, in any combination. The umbilicus may further comprise a handle connection end converter located within the handle connection end, wherein the handle connection end converter converts a plurality of electrical signals into a plurality of light signals for transmission through the circuitry to the controller connection end. The umbilicus may further comprise a controller connection end converter located within the controller connection end, wherein the controller connection end converter converts a plurality of light signals into a plurality of electrical signals for transmission to the controller. The umbilicus may further comprise a light source located within a housing of the handle connection end.

In another example, a method for attaching a reusable umbilicus to first and second medical device handles may comprise attaching a handle connection end of an umbilicus to the first medical device handle; disconnecting the handle connection end of the umbilicus from the first medical device handle; and attaching the handle connection end of the umbilicus to the second medical device handle.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIG. 1 depicts an exemplary medical device of this disclosure.

FIG. 2 depicts an exemplary medical system of this disclosure.

FIG. 3 depicts an exemplary umbilicus of this disclosure.

FIG. 4 depicts an exemplary handle connection end of an exemplary umbilicus of this disclosure.

FIG. 5 depicts an exemplary flexible circuit for use within an exemplary umbilicus of this disclosure.

FIG. 6 depicts exemplary fluidics tubing attached to an exemplary umbilicus of this disclosure.

### DETAILED DESCRIPTION

As discussed in the Background, a medical device comes into contact with a patient during an endoscopy procedure. After the endoscopy procedure, the medical device may be cleaned, or disposed of, to avoid cross-contamination between patients. However, improper sterilization of the medical device may result in cross-contamination between patients. Additionally, disposing of a medical device after every procedure creates large amounts of waste and higher costs. As a result, a need exists for improved devices, systems, and methods for avoiding cross-contamination between endoscopy patients.

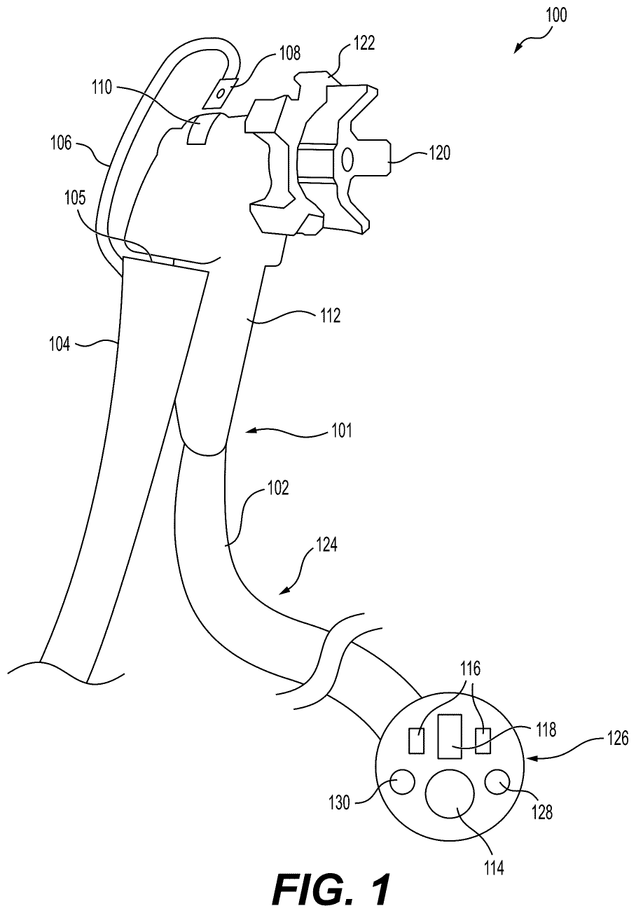

FIG. 1 depicts an exemplary medical device 100 that includes two parts that releasably connect: an umbilicus 104 (which may be reusable) and a second part 101 (which may be single-use and/or otherwise disposable). The second part 101 may be comprised of a handle 112 and an insertion portion 124. Although a cholangioscope may be referenced herein as the medical device 100, it will be appreciated that the disclosure also encompasses duodenoscopes, endoscopes, bronchoscopes, gastroscopes, endoscopic ultrasound (EUS) scopes, colonoscopes, ureteroscopes, bronchoscopes, laparoscopes, cystoscopes, aspiration scopes, sheaths, catheters, similar devices, or any other medical device that may be used in combination with an umbilicus/cable that transmits electrical signals, power, etc. A reference to a cholangioscope herein should be understood to encompass any of the above medical devices.

The handle 112 may have one or more actuators/control mechanisms. The control mechanisms may provide control over a steerable section, an elevator, and/or imaging functions, and/or may allow for the provision of air, water, suction, etc. For example, the handle 112 may include control knobs 120, 122 for left, right, up, and/or down control of a steerable section of a sheath or shaft 102. For example, one of the knobs 120, 122 may provide left/right control of the steerable section, and the other of the knobs 120, 122 may provide up/down control of the steerable section. The handle 112 may further include one or more locking mechanisms (e.g., knobs or levers) for preventing steering and/or braking of the steerable section in at least one of an up, down, left, or right direction. The handle 112 may include an elevator control lever (e.g., if medical device 100 is a duodenoscope). The elevator control lever may raise and/or lower an elevator, via a connection between the lever and an actuating wire that extends from the lever, through the shaft 102, to the elevator.

The insertion portion 124 includes a sheath or shaft 102 and a distal end 126. The distal end 126 includes an imaging device 118 (e.g., a camera, such as a CCD camera, or other image sensors, including analog or digital sensors), one or more light sources 116 (e.g., an LED or an optical fiber), a working channel 114, and channels 128 and 130 for, for example, suction and irrigation of fluids, respectively. The distal end 126 is shown as distal/front facing, with its components facing distally. The distal end 126, however, may be side-facing. That is, the imaging device 118 and the lighting source(s) 116, and optionally one or more other distal end components, including the openings of channels 114, 128, and 130, may face radially outward, perpendicularly, approximately perpendicularly, or otherwise transverse to a longitudinal axis of the shaft 102 and the distal end 126. Additionally or alternatively, the distal end 126 may include one or more imaging devices 118 that face in more than one direction. For example, a first imaging device 118 may face radially outward, and a second imaging device 118 may face distally (approximately parallel to a longitudinal axis of the distal end 126/shaft 102).

A distal portion of the shaft 102 that is connected to the distal end 126 may have a steerable section. The steerable section may be, for example, an articulation joint. The shaft 102 and the steerable section may include a variety of structures which are known or may become known in the art. The suction channel 128 is a lumen that extends through the insertion portion 124 and provides suctioning functionality at the distal end 126. The irrigation channel 130 is a lumen that extends through the insertion portion 124 and provides irrigation functionality at the distal end 126, for cleaning the imaging device 118 or clearing a treatment site for better visualization.

The umbilicus 104 releasably attaches to the handle 112 for purposes of connecting the handle 112, shaft 102, distal end 126, and components at the distal end 126 to sources of, for example, power, imaging device control, image processing, light, light control, and/or display/viewing equipment. The umbilicus 104 includes the cabling/wires and electronics to support the transmission of power and control signals from a control box (not shown) to the second part 101, and particularly to any of a variety of image sensors (e.g., imaging device 118) and light sources 116 (such as LEDs). The cabling also includes cables/wires and electronics to support the transmission of image data from the imaging device 118 to the control box for processing.

The umbilicus 104 may include a flexible tether 106 that branches off from a main part of the umbilicus 104. For example, the tether 106 may face radially outward, perpendicularly, approximately perpendicularly, or otherwise transverse to a longitudinal axis of the main part of the umbilicus 104. The tether 106 includes at least one button 108 at the free end of the tether 106 furthest from the main part of the umbilicus 104. The button 108 electrically connects to other cabling/wires in the main part of the umbilicus 104 via cables/wires within the tether 106. When pressed, the button 108 controls an image/video capture functionality of the medical device 100 or any other desired functionality.

In some embodiments, the free end of the tether 106 includes an engagement feature for securably, yet releasably, engaging the handle 112. In this way, the user may securely attach the free end of tether 106, and its button 108, at a position on the handle 112 that is ergonomic for ease of use during a procedure. In some embodiments, the handle 112 may include multiple different positions for attachment of the free end of the tether 106 to the handle 112, so that the user can select an optimal, desired position for use. Alternatively, the free end of the tether 106 and the button 108 may remain unattached from the handle 112, for placement at any desired location for use.

In some embodiments, the free end of the tether 106 may be shaped (for example, a rectangular outer profile) or have a protrusion to engage a correspondingly-shaped portion, such as channel 110, in the body of the handle 112. The engagement can be via a snap-fit, a friction-fit, relative sliding, or other engagement that securely holds the tether 106 to the handle 112, yet permits release after use. As another example, in some embodiments, the free end of the tether 106 includes a magnet that attaches to the handle 112 via a metal feature embedded into the handle 112. For example, a small piece of metal may be in the interior of the body of the handle 112 at a location suitable for placement of the button 108. The exterior of the body of the handle 112 may have a suitable indicator/mark for the user to recognize for placement of the free end of the tether 106 and its magnet. Alternatively, in some embodiments, the handle 112 includes a magnet that attaches to a metal feature at the free end of the tether 106.

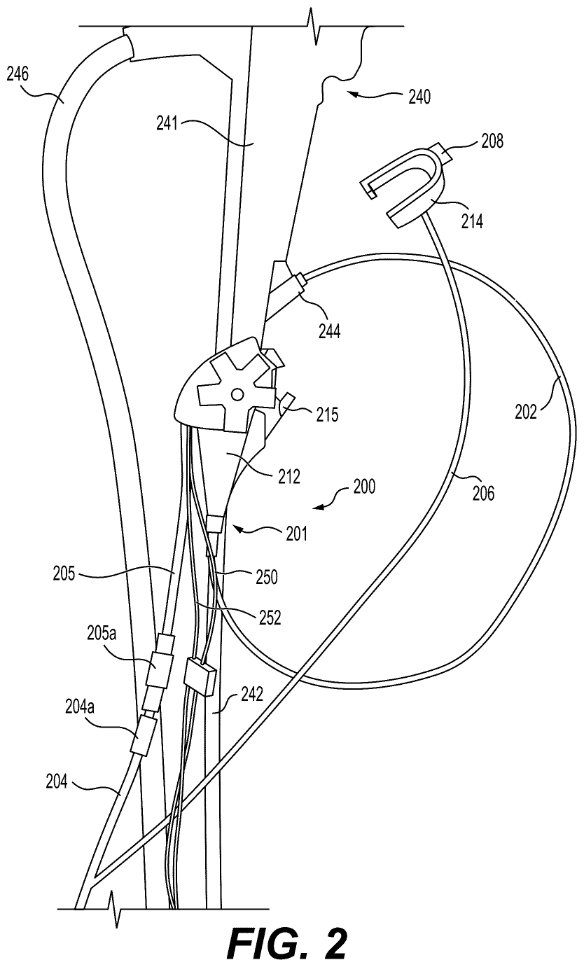

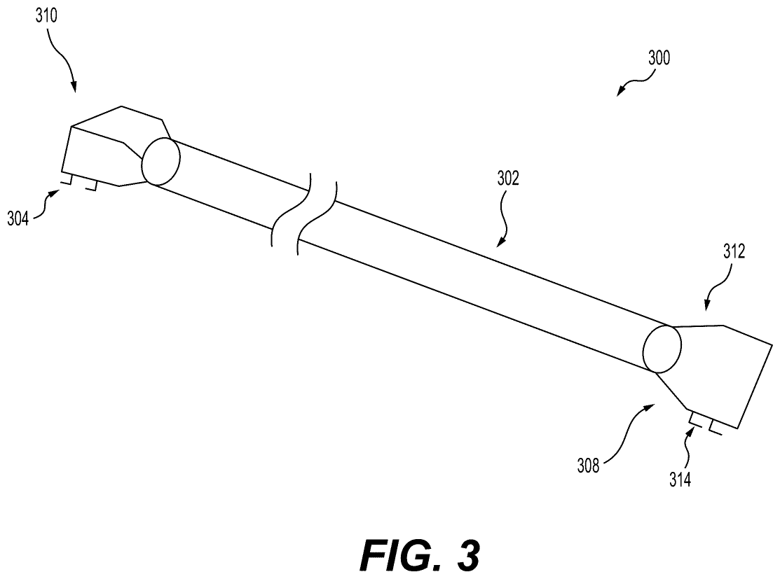

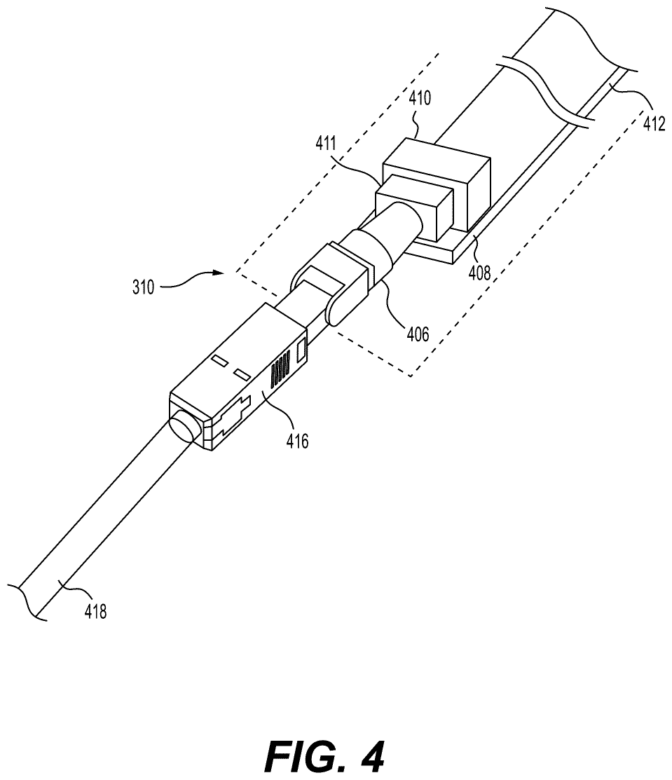

In some embodiments, the umbilicus 104 may connect to the handle 112 at a connection point 105, via any suitable electrical and physical/mechanical connection that will securely yet releasable attach the umbilicus 104 to the handle 112 and permit the transmission of electrical signals, power, etc. between the umbilicus 104 and the second part 101 of the medical device 100. The physical connection may include a mating physical connection. In one aspect, the umbilicus 104 may include a female connector and the handle 112 may include a male connector, as will be described in connection with FIG. 2. In another aspect, handle 112 may include a female connector and the umbilicus 104 may include a male connector. The umbilicus 104 and the handle 112 may include any suitable electrical connector, including pins, plugs, ports, solder connections, etc. The discussion of FIGS. 3 and 4 below includes additional detail regarding an embodiment of electrical connections of the umbilicus 104 and the handle 112.

In use, an operator may connect the umbilicus 104 to the handle 112, and connect the free end of tether 106 to an ergonomically-desirable position on the handle 112. The operator then may insert at least a portion of the shaft 102 into a body lumen of a subject. The distal end 126 may be navigated to a procedure site in the body lumen. The operator may insert an accessory device (not shown) into a port (not shown) of the handle 112, and pass the accessory device through the shaft 102 via the working channel 114 to the distal end 126. The accessory device may exit the working channel 114 at the distal end 126. The operator may use the accessory device to perform a medical procedure. Upon conclusion of the procedure, the accessory device may be removed from the working channel 114, and the shaft 102 removed from the body lumen. The operator then may disconnect the umbilicus 104 from the handle 112, and disconnect the free end of tether 106 from the handle 112. The second part 101 of the medical device 100, in at least some embodiments, may be disposed of. And in some embodiments, the umbilicus 104 may be cleaned and re-used with another medical device in a subsequent procedure.

FIG. 2 depicts a medical system that includes multiple medical devices, including an endoscope or duodenoscope 240 (also referred to as a mother scope) and a medical device 200 that is the same as the medical device 100 except as discussed below. As is known in the art, the medical device 200 may be a cholangioscope that is used in combination with a duodenoscope for use in, for example, an endoscopic retrograde cholangiopancreatography (ERCP) procedure.

Like the medical device 100, the medical device 200 includes a handle 212 (with an access port 215 that was not visible in FIG. 1), a shaft 202, and an umbilicus 204. These parts are similar to the corresponding parts described in FIG. 1.

Umbilicus 204 includes a female connector 204a configured to releasably connect to a male connector 205a of an extension part 205. The extension part 205 is permanently attached to and extends from the handle 212. The extension part 205 includes a tube with wires/cables that extend from the interior of the handle 212 and connect to the male connector 205a. As a result, the female connector 204a and the male connector 205a provide a connection point between the handle 212 and the umbilicus 204. The female connector 204a and the male connector 205a may be any suitable known connector of electrical wires/cables, such as pins, plugs, ports, solder connections, etc.

Additionally, as shown in FIG. 2, the tether 206 is similar to the tether 106, except that the tether 206 includes a c-shaped connector 214 at the free end of the tether 206 furthest from the main part of the umbilicus 204. The c-shaped connector 214 includes a button 208 that is similar to button 108. For example, the button 208 is connected to a wire that goes through the tether 206 and into the main body of the umbilicus 204. When pressed, the button 208 may be configured to control an image/video capture functionality of the medical device 200.

Medical device 200 may include flexible tubes 250 and 252 for respectively providing suctioning functionality and irrigation functionality at the distal end, for cleaning the imaging device or clearing a treatment site for better visualization. For example, the irrigation tube 252 includes a lumen that extends into the handle 212 and couples to the irrigation channel of the insertion portion 224 to provide irrigation functionality at the distal end. Likewise, the suction tube 252 includes a lumen that extends into the handle 212 and couples to the aspiration channel of the insertion portion 224 to provide suctioning functionality at the distal end.

The duodenoscope 240 may be a standard duodenoscope. For example, the duodenoscope 240 may include a handle 241 with an access port 244. The access port 244 may accept shaft 202 for insertion into a subject. For example, the shaft 202 may be inserted through the access port 218, through the shaft 242, and into the subject.

The handle 212 of the medical device 200 may connect to the duodenoscope handle 241 via a strap or other like connector, as is known in the art. The c-shaped connector 214 may connect to the duodenoscope handle 241 at any desired longitudinal position along the duodenoscope handle 241. For example, the c-shaped connector 214 may be connected at any ergonomic position of the duodenoscope handle 241. In other embodiments, the c-shaped connector may be connected at a position on handle 212. The c-shaped connector 214 may be made out of a semi-flexible material that is configured to attach to the duodenoscope handle 241. Additionally, the ends of the “c” of the c-shaped connector 214 are capable of flexing in and out to secure to the duodenoscope handle 241. In one aspect, the c-shaped connector 214 may have a dimension and a flexibility to friction fit to the duodenoscope handle 241. In another aspect, the c-shaped connector 214 may attach to the duodenoscope handle 210 via hook and loop fasteners (e.g., Velcro), or other suitable means to securely attach and release the c-shaped connector 214 to the duodenoscope handle 241. In another aspect, the c-shaped connector 214 may include a magnet that attaches to the duodenoscope handle 241 via a magnetic metal strip that extends longitudinally on the outer part (or the inside) of the duodenoscope handle 241. Alternatively, the c-shaped connector 214 and its button 208 may remain unattached from the duodenoscope handle 241 during use, and may instead be placed at any desired location.

Embodiments of a reusable umbilicus and a disposable second part (e.g., handle and insertion portion) of the medical device can provide one or more of the following benefits. First, disposable second part minimizes cross-contamination between subjects due to insufficient cleaning of reusable medical devices, including scopes. For example, one or more areas of an endoscope may be insufficiently sterilized during cleaning, which may result in cross-contamination between subjects. However, disposing the parts of the endoscope that come in contact with a subject, while reusing the components (e.g., the umbilicus) of the endoscope that may not come in contact with the subject, minimizes the potential for cross-contamination.

Second, a reusable umbilicus and a disposable second part may reduce or eliminate the costs and logistics associated with cleaning reusable endoscopes. Though a reusable umbilicus may need cleaning, it may require less rigorous cleaning procedures because it does not contact the subject. Third, a reusable umbilicus and a disposable second part may facilitate a connection to the controller after the connection of a cholangioscope to a duodenioscope. For example, the second part of the cholangioscope may be fastened to the duodenoscope, as shown in FIG. 2, before connecting the umbilicus to the second part. This sequence can be easier to carry out because the umbilicus does not get in the way of the cholangioscope to duodenoscope connection.

Fourth, a reusable umbilicus and a disposable second part may resulting in a lower overall cost, especially as compared to fully disposable, medical devices/scopes. For example, including a button for controlling video/image capture in the reusable umbilicus may reduce the overall cost. Additionally, for example, incorporating the receiving circuitry for light in the umbilicus may reduce the number of copper conductors and overall cost, as well as reduce the radiated emissions of the system. Moreover, placing the LED in the umbilicus may result in lower overall cost and keeping the heat from the LED away from the body of a subject.

Fifth, a reusable umbilicus and a disposable second part may also reduce the weight of a single use device by up to approximately 50%, which may aid in increasing sustainability and reducing the environmental impact. Additional benefits may include potentially reducing the packaging size for storage, as well as reduced overall shipping costs.

FIG. 3 depicts an embodiment of an umbilicus 300 that could be used for umbilicus 104 or umbilicus 204 in the embodiments shown in FIGS. 1 and 2. The umbilicus 300 may comprise the same (or similar) elements as the umbilicus 104 and the umbilicus 204 of FIGS. 1 and 2 respectively. For example, the umbilicus 300 may include a tether like those shown in FIGS. 1 and 2 (though not shown in FIG. 3). The umbilicus 300 connects to part of a medical device (e.g. second part 101 of medical device 100) at a handle connection end 310 and also connects to a controller (e.g., a control box) at a controller connection end 312. The umbilicus 300 also includes electrical wires and/or optical circuitry extending within a main, elongate, flexible tube 302 between the handle connection end 310 and the controller connection end 312. The electrical wires and/or optical circuitry may include cables, wires, optical fibers, and/or illumination fibers for transmitting electrical signals, such as image sensor data, imager instruction signals, and power between the medical device and the controller. The electrical wires and/or optical circuitry also may transmit light and/or light instruction signals from the controller to the medical device.

The handle connection end 310 includes solder connections 304 (e.g., in the form of bumps, balls, or the like) for releasable, electrical connection to corresponding electrical connections at the handle of the medical device, which in turn may electrically connect to a flex circuit within the handle. In some embodiments, the solder connections 304 may directly connect, through wires for example, to a flexible circuit housed within the handle connection end 310. These flexible circuits may provide image processing (for example, amplification, filtering, etc.), power, and/or electricity.

The controller connection end 312 includes solder connections 314 (e.g., in the form of bumps, balls, or the like) for releasable, electrical connection to corresponding electrical connections at the controller (e.g. a control box; not shown), which in turn electrically connect to control/power/light components and circuitry in the controller. In some embodiments, the solder connections 314 may directly connect, through wires for example, to a flexible circuit housed within the controller connection end 312. The flexible circuit may provide image processing (for example, amplification, filtering, etc.), power, and/or electricity.

The distal end of the medical device (e.g., medical device 100) may include an imaging device (e.g., imaging device 118) that captures image data of a subject. The imaging device transmits such image data as electrical signals to the handle (e.g., handle 112), where a flex circuit in the handle may perform basic processing of the image data. The image data is then transmitted as electrical signals to the handle connection end 310 of the umbilicus 300, via the connection between solder connections 304 and the corresponding connections in the handle. Further processing may be performed in one or more flex circuits within umbilicus 300 as that image data is transmitted, for example via the electrical wires or optical circuitry within the tube, to the controller connection end 312. In some embodiments, a converter within the handle connection end may convert electrical signals to light signals, for transmission through optical circuitry to the controller connection end 312, which also may have a converter to convert the light signals (carrying image data, for example) back to electrical signals for transmission to the controller. The controller receives the electrical signals at the controller connection end 312 and processes the electrical signals (e.g., in a processor of the controller) and causes display of the image on a monitor, for example.

The electrical wires and/or optical circuitry also transmit light and/or light signals and power from the controller to the handle of the medical device. There are at least two pathways/embodiments for the light transmission that keep the source of the light away from the distal end of the medical device. This reduces heat, and its attendant disadvantages, from that distal end.

In a first embodiment, the umbilicus 300 includes a light source, such as an LED, within a housing of the handle connection end 310. The controller sends power signals and/or control signals through the optical circuitry of the tube 302 to the light source. The light source receives the signals and provides light. Through the connection of the handle connection end 310 to the handle, the light transmits into a fiber optic cable to a distal end of a shaft of the medical device.

In a second embodiment, the controller includes the light source (e.g., LED). The controller sends power signals and/or control signals to the light source within the controller. The light source then sends light into the controller connection end 312 of the umbilicus 300, through the optical circuitry (e.g. fibers) within the tube 302, and to the handle connection end 310, where the light is transmitted via a fiber optic connector to fiber optics within the handle and the shaft of the medical device, to the distal end of the device.

FIG. 4 depicts an embodiment of part of a handle connection end of an umbilicus (denoted by the parts within the dashed lines) and part of a medical device (such as part of the handle) that connects to the umbilicus. The umbilicus can include many of the same (or similar) elements as discussed in connection with FIG. 3. The handle connection end of the umbilicus includes an umbilicus connector 406, a flexible printed circuit board (PCB) 408, a heat sink 410, and a light source 411 (such as an LED), and a flexible PCB extension 412. The heat sink 410 has a thermal conductor for absorbing heat, and the flexible PCB 408 includes a copper plate for the heat sink 410. The flexible PCB extension 412 extends from the flexible PCB 408 and into the tube of the umbilicus, carrying light signals from the controller.

The umbilicus connector 406 is configured to removably attach to an end of a handle connector 416 of a handle of the medical device, which may be a single use device. The other end of the handle connector 416 is attached to one or more optical fibers 418 (e.g., fibers for illumination) that extend through the handle and the shaft of the medical device to the distal end. In operation, the light source 411 transmits light through the umbilicus connector 406 to the handle connector and the fibers 418, and to the distal end of the shaft.

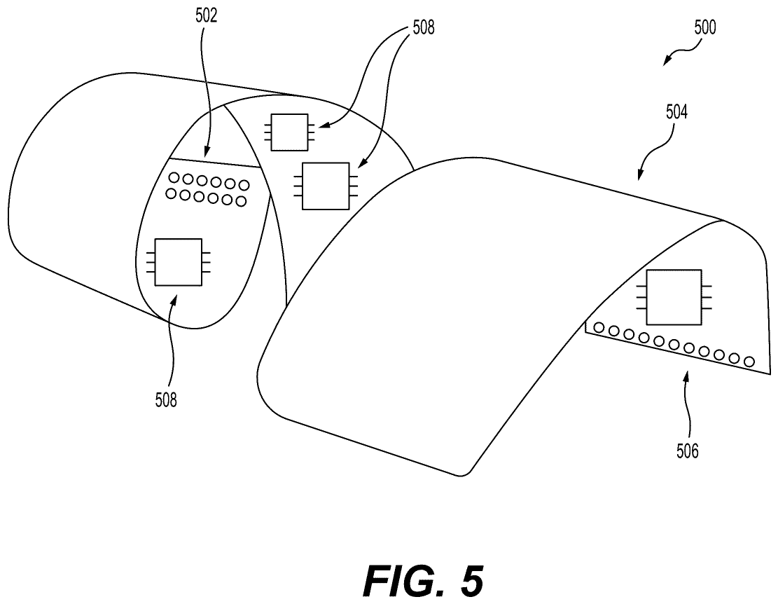

FIG. 5 illustrates a flexible circuit 500 for use within an umbilicus, such as any of the umbilici shown and described herein. The flexible circuit 500 is thin and can have a coiled/spiral/rolled shape or configuration (shown in FIG. 5), a second substantially planar, flat configuration (not shown) when not in the first configuration, and any other shape desired based on its flexibility. The flexible circuit 500 may be included in an end of the umbilicus that is closest to the handle of the medical device when connected.

The flexible circuit includes umbilicus connector connections 502, umbilicus cable connections 506, an outer layer 504, and flexible circuits 508 and other circuitry within the outer layer 504. The umbilicus connector connections 502 attach to the electrical connections, for example the solder connections 304, which electrically connect to corresponding connections of the handle. The umbilicus cable connections 506 attach to the cable within the umbilicus. Each of the connections 502 and 506 may include a pattern of solder connections, such as solder bumps.

The outer layer 504 is an insulative layer that provides a ground plane for shielding and impedance control. The outer layer 504 also covers the circuits 508 and other circuitry within the flexible circuit 500. The circuits 508 may be staggered inside the outer layer 504. The circuits 508 may include a microcontroller, as well as various repeater or driver circuits. The microcontroller provides communications between the buttons of the medical device and the umbilicus. The microcontroller also communicates with an EEPROM (electrically erasable programmable read-only memory) that is located in the handle of the medical device. The EEPROM stores the data required for operation of the camera, as well as provides a secure area for reuse prevention. The repeater circuits may be analog and/or LVDS (low-voltage differential signaling), depending on the type of camera that is used by the medical device. For example, a digital camera may use a LVDS repeater, where an analog camera would use an analog amplifier/cable driver. Other circuits may include LED and driver circuits for illumination, or LED transceiver circuits for fiber data transmission.

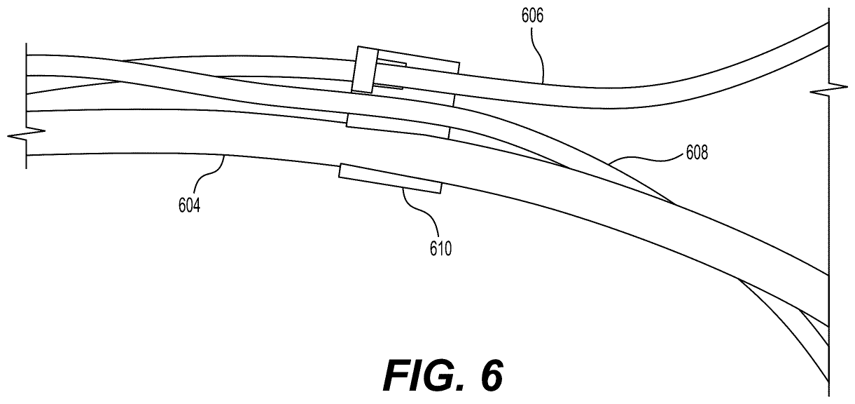

FIG. 6 illustrates fluidics tubing 606, 608 attached to an umbilicus 604, such as any of the umbilici shown and described herein. The fluidics tubing 606, 608 may include fluid management tubing (e.g., for irrigation and suction) as described in connection with tubes 250 and 252 in FIG. 2. The fluidics tubing 606, 608 may be attached to the umbilicus 604 via a clip 610. In some embodiments, the clip 610 is molded into the outer surface of the umbilicus 604. The clip 604 may allow the fluidics tubing 606, 608 to removably attach to the umbilicus 604, for easier management of the various tubing.

In some embodiments, the fluidics tubing 606, 608 may be placed through one or more dedicated lumens inside of the umbilicus 604. For example, the umbilicus may have one or two lumens, open at each end of the umbilicus 604, for inserting the tubing 606, 608, prior to a procedure. After the procedure, the tubing 606, 608 may be removed from the umbilicus 604. The tubing 606, 608 is fixed to the disposable second part (including the handle) and is therefore disposable, which reduces the risk of cross-contamination.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. A medical device, comprising:
a first part having a handle, a shaft attached to the handle, and a distal assembly at a distal end of the shaft; and
an umbilicus releasably attached to the handle of the first part.

2. The medical device of claim 1, wherein the umbilicus includes a tether that branches off from a main body of the umbilicus.

3. The medical device of claim 2, wherein the tether includes at least one button at an end of the tether furthest from the main body of the umbilicus, wherein the at least one button is configured to control an imaging function of the medical device.

4. The medical device of claim 2, wherein an end of the tether furthest from the main body includes an engagement feature for securably and releasably engaging the handle of the first part.

5. The medical device of claim 4, wherein the engagement feature is configured to securably and releasably engage multiple different positions of the handle.

6. The medical device of claim 4, wherein the tether has a shape or a protrusion to engage a correspondingly-shaped portion in a body of the handle, wherein the protrusion is a snap-fit engagement, a friction-fit engagement, or a relative sliding engagement that securely holds the tether to the handle and permits release after use.

7. The medical device of claim 4, wherein the engagement feature includes a magnet that attaches to the handle via a metal feature of the handle.

8. The medical device of claim 4, wherein the engagement feature includes a c-shaped connector.

9. The medical device of claim 1, wherein the umbilicus is configured to connect to the handle at a connection point via a mating, physical and electrical connection that securely and releasably attaches the umbilicus to the handle.

10. The medical device of claim 1, wherein the umbilicus includes a handle connection end, wherein the handle connection end includes an umbilicus connector, a flexible printed circuit board, and a heat sink.

11. The medical device of claim 10, wherein the umbilicus includes a handle connection end converter located within the handle connection end, wherein the handle connection end converter converts a plurality of electrical signals into a plurality of light signals for transmission through circuitry in a main body of the umbilicus to a controller connection end.

12. The medical device of claim 10, wherein the umbilicus includes a controller connection end converter located within a controller connection end, wherein the controller connection end converter converts a plurality of light signals into a plurality of electrical signals for transmission to a controller.

13. The medical device of claim 1, the medical device further comprising:
one or more fluidics tubing removably attached to the umbilicus via a clip, wherein the one or more fluidics tubing is fixed to the handle.

14. The medical device of claim 1, wherein the umbilicus includes a flexible circuit located in an end of the umbilicus that is closest to the handle when connected.

15. The medical device of claim 14, wherein the flexible circuit includes at least one umbilicus connector connection attached to at least one electrical connection of the handle, at least one umbilicus cable connection attached to a cable within the umbilicus, and an outer layer that includes at least one additional flexible circuit.

16. An umbilicus, comprising:
a main body that includes circuitry for transmitting power, imaging signals, and light or light signals;
a handle connection end at a first end of the main body, wherein the handle connection end is configured to releasably connect to a medical device handle; and
a controller connection end at a second end of the main body opposite the handle connection end, wherein the controller connection end is configured to releasably connect to a controller.

17. The umbilicus of claim 16, further comprising:
a handle connection end converter located within the handle connection end, wherein the handle connection end converter converts a plurality of electrical signals into a plurality of light signals for transmission through the circuitry to the controller connection end.

18. The umbilicus of claim 16, further comprising:
a controller connection end converter located within the controller connection end, wherein the controller connection end converter converts a plurality of light signals into a plurality of electrical signals for transmission to the controller.

19. The umbilicus of claim 16, further comprising:
a light source located within a housing of the handle connection end.

20. A method for attaching a reusable umbilicus to first and second medical device handles, the method comprising:
attaching a handle connection end of an umbilicus to the first medical device handle;
disconnecting the handle connection end of the umbilicus from the first medical device handle; and
attaching the handle connection end of the umbilicus to the second medical device handle.
